(19) United States
(12) Patent Application Publication
(10) Pub. No.: US 2016/0256496 A1
(43) Pub. Date: Sep. 8, 2016

(54) PHARMACEUTICAL COMPOSITION AND METHOD FOR THE TREATMENT OF ORTHOPEDIC PATHOLOGIES

(71) Applicant: SCARCELL THERAPEUTICS, Paris (FR)

(72) Inventors: Bruno GOGLY, Hondevilliers (FR); Antoine LAFONT, Paris (FR); Bernard COULOMB, Igny (FR)

(21) Appl. No.: 15/061,606

(22) Filed: Mar. 4, 2016

Related U.S. Application Data
(62) Division of application No. 14/434,963, filed on Apr. 10, 2015, now abandoned.

(30) Foreign Application Priority Data
Dec. 11, 2009 (FR) 09 58887

(51) Int. Cl. A61K 35/33

(52) U.S. Cl. CPC A61K 35/33

(57) ABSTRACT

The present invention relates to a gingival fibroblast-derived product and its use in methods for the prevention or treatment of orthopedic pathologies in an individual.

### CROSS-REFERENCE TO RELATED APPLICATION

This application is a divisional of U.S. patent application Ser. No. 14/434,963, filed Apr. 10, 2015, which is a 371 of International application PCT/FR2010/052670, filed Dec. 10, 2010, all of said applications incorporated herein by reference.

### FIELD OF THE INVENTION

The present invention relates to the compositions and methods for the prevention and treatment of orthopedic pathologies.

### TECHNICAL BACKGROUND

The prevalence of osteoarticular pathologies is considered to be of about 25% of the population of industrialized countries. Orthopedic pathologies, in particular osteoarticular pathologies, target musculoskeletal tissues, such as bones, cartilage and synovial membranes, ligaments, tendons and muscles, which are deteriorated in these pathologies. There are numerous causes of deteriorations such as traumas, aging, mechanical wearing, or inflammation. Examples of orthopedic pathologies are osteoarthritis and rheumatoid arthritis.

Among the various strategies considered for treating orthopedic pathologies, cellular therapy appears to be a promising field.

In this regard, mesenchymal stem cells (MSCs), non-hematopoietic progenitor cells which can be found in various adult tissues, such as bone marrow, adipose tissue or derma, and which can give yield to some conjunctival skeletal tissues, such as bones and cartilage, have been used in the frame of the treatment of orthopedic pathologies. In particular, it has been attempted to treat arthritic diseases with MSCs (Chen & Tuan (2008) Arthritis research & Therapy 10:223-234).

However, it appears MSCs engraft rather poorly to articular cartilage within the frame of osteoarthritis treatment (Chen & Tuan (2008) Arthritis research & Therapy 10:223-234). Besides, contrasted results have been reported for the treatment of rheumatoid arthritis, as some authors mention that MSCs would not be helpful to improve the status of patients (Djouad et al. (2005) Arthritis and Rheumatism 52:1595-1603). It has been suggested that these contrasted results could be a consequence of the absence of a clear definition of MSCs and of their heterogeneity.

Accordingly, there is a need for an alternative to MSCs which would be more effective in treating orthopedic pathologies.

Gingival fibroblasts are adult cells of mesenchymal origin which are capable of migrating, adhering and proliferating within the soft connective tissues of the gum. They thus maintain the integrity of the gingival tissue which is exposed to numerous aggressions, such as mechanical stresses, bacterial infections, or pH and temperature variations. Gingival fibroblasts are in particular described in Gogly et al. (1997) Clin. Oral Invest. 1:147-152; Gogly et al. (1998) Biochem. Pharmacol. 56:1447-1454; and Ejeil et al. (2003) J. Periodontol. 74:188-195.

Depending on environmental conditions, gingival fibroblasts are capable to modulate their phenotype, and to respond by proliferating, migrating, and by synthesizing or degrading matrix components or matrix-related enzymes.

Gingival fibroblasts synthesize collagens (e.g. types I, III, V, VI, VII, XII), elastic fibers (oxytalan, elaunin and elastin), proteoglycans and glycosaminoglycans (e.g. decorin and biglycan), and glycoproteins (e.g. fibronectin and tenascin). Simultaneously, depending on the context, gingival fibroblasts synthesize enzymes that are able to degrade macromolecular compounds (matrix metalloproteinases; MMPs), but also enzymes inhibiting active forms of MMPs (Inhibitors of metalloproteinases; TIMPs). Gingival fibroblasts are thus important actors of extracellular matrix remodeling.

### SUMMARY OF THE INVENTION

The present invention follows on from the unexpected finding, by the inventors, that gingival fibroblasts cultured with human chondrocytes, myocytes or osteoblasts, stimulated by a pro-inflammatory cytokine, could inhibit the MMP activity secreted by these cells. This demonstrates that gingival fibroblasts are capable of inhibiting MMP activity in an environment similar to that of orthopedic pathologies, in particular inflammatory orthopedic pathologies.

The present invention thus relates to a method for the prevention or treatment of orthopedic pathologies of an individual, wherein a prophylactically or therapeutically effective amount of a gingival fibroblast-derived product is administered to the individual.

The present invention also relates to a gingival fibroblast-derived product for its use in the prevention or treatment of orthopedic pathologies of an individual.

The present invention also relates to a gingival fibroblast-derived product for its use in the manufacture of a medicament intended for the prevention or treatment of orthopedic pathologies of an individual.

### DETAILED DESCRIPTION OF THE INVENTION

As intended herein, an orthopedic pathology according to the invention is a pathology targeting musculoskeletal tissues, namely, in particular, bones, cartilage, synovial membranes, ligaments, tendons and muscles, which can notably be deteriorated. Thus, the orthopedic pathology according to the invention is preferably selected from the group consisting of an osteoarticular orthopedic pathology, a muscular orthopedic pathology, a ligamentary orthopedic pathology and a tendinous orthopedic pathology.

The orthopedic pathology according to the invention may notably be a consequence of traumas, aging, mechanical wearing, or inflammation of a musculoskeletal tissue as defined above.

In particular, the orthopedic pathology according to the invention is an osteoarticular, more particularly articular, notably inflammatory, pathology. In a particularly preferred embodiment, the orthopedic pathology according to the invention is rheumatoid arthritis or osteoarthritis.

Preferably, the individual according to the invention is a mammal, more preferably a human.

In a particular embodiment, gingival fibroblasts according to the invention comprise at least 75, 80, 90, 95 or 100% of gingival fibroblasts as such, that is gingival fibroblasts which have not undergone a differentiation, in particular into cells having an osteogenic phenotype.

The gingival fibroblasts can also comprise progenitor cells, preferably less than 25, 20, 15, or 5%

In another particular embodiment, the gingival fibroblasts may for instance be those described in Fournier B P et al. (2010) Tissue Eng. Part A. 16(9):2891-9.

Procedures for taking, culturing and preserving gingival fibroblasts are well known to one skilled in the art and are particularly described in Naveau et al. (2006) J. Periodontol. 77:238-47 and in Gogly et al. (2007) Arterioscler. Thromb. Vasc. Biol. 27:1984-90.

Advantageously, gingival fibroblasts are easily sampled and cultured. Besides, gingival fibroblasts possess a high growth speed.

Preferably, the gingival fibroblasts used in the method according to the invention are autologous, that is they are taken from the individual to whom the gingival fibroblast-derived product is intended to be administered. Advantageously, gingival fibroblasts provide for an almost limitless source of autologous fibroblasts. However, the gingival fibroblasts can also be allogenic, that is taken from another individual of the same species, or heterologous, that is taken from another individual of another species.

As intended herein, the expression “gingival fibroblast-derived product” relates to any product which can be obtained from gingival fibroblasts in themselves or which contains gingival fibroblasts secretions.

For example, it is preferred that the gingival fibroblast-derived product is selected from the group consisting of gingival fibroblast whole cells, in particular live gingival fibroblast whole cells, a gingival fibroblast culture, a gingival fibroblast extract, and a gingival fibroblast conditioned medium.

The gingival fibroblast extract according to the invention can be obtained by any cell fragmentation method known in the art. In particular, the gingival fibroblast extract according to the invention can be a membrane extract, a cytoplasmic extract or a nuclear extract.

The gingival fibroblast conditioned medium according to the invention relates to any medium, such as a liquid cell culture medium (for instance the “Dulbecco's Modified Eagle Medium”, or a culture medium without serum), which has been contacted by gingival fibroblasts, in particular for a time sufficient for the gingival fibroblasts to have secreted in the medium.

Administration of the gingival fibroblast-derived product as defined above to the individual, preferably near or at a corporal site to be treated, can proceed by any method known in the art. It is nevertheless preferred that the gingival fibroblast-derived product is administered by injection at a site of orthopedic defect. As intended herein, a site of orthopedic defect relates to any pathological area of a musculoskeletal tissue as defined above.

Preferably, the method of prevention or of treatment according to the invention comprises or consists of the following steps:
taking gingival fibroblasts from an individual;
culturing the gingival fibroblasts;
obtaining a gingival fibroblast-derived product as defined above from the cultured gingival fibroblasts;
administering the gingival fibroblast-derived product to the individual.

When the gingival fibroblast-derived product consists of or comprises whole cells, these cells can be administered within the frame of a cellular therapy.

### DESCRIPTION OF THE FIGURES

FIG. 1: Quantification of MMP1 by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

FIG. 2: Quantification of MMP3 by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

FIG. 3: Quantification of MMP7 by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

FIG. 4: Quantification of MMP9 by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

FIG. 5: Quantification of TIMP1 by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β°hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

FIG. 6: Quantification of the MMP1/TIMP1 complex by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

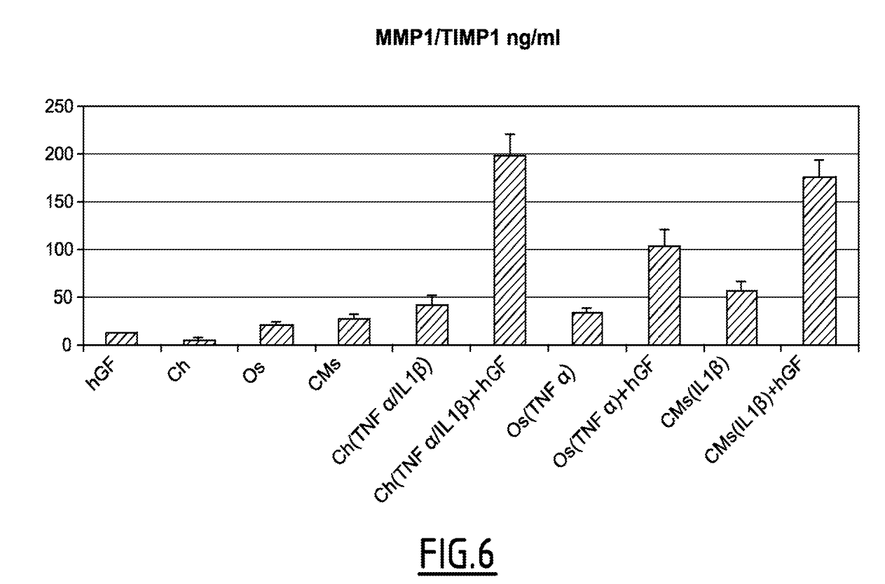

FIG. 7: Quantification of the MMP3/TIMP1 complex by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

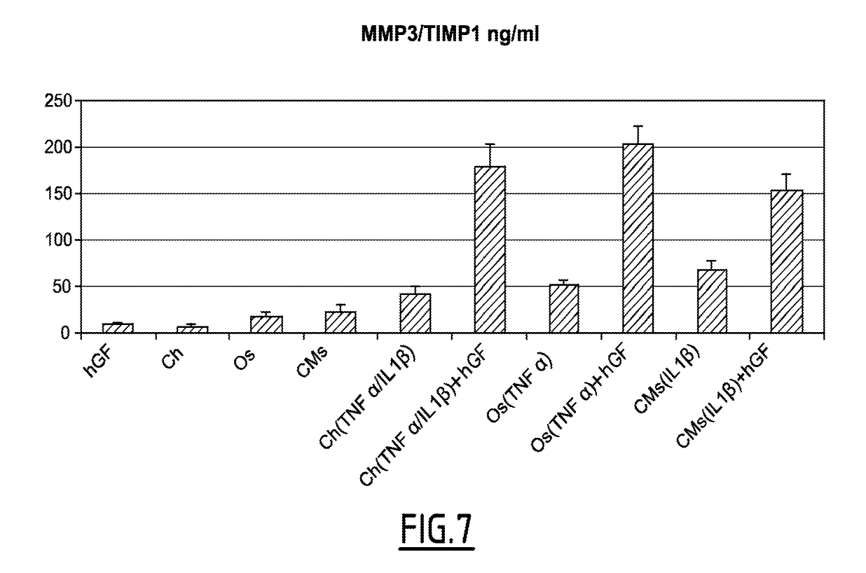

FIG. 8: Quantification of the MMP7/TIMP1 complex by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β)) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β)) or co-cultured with hGF (CMs(IL1β)+hGF).

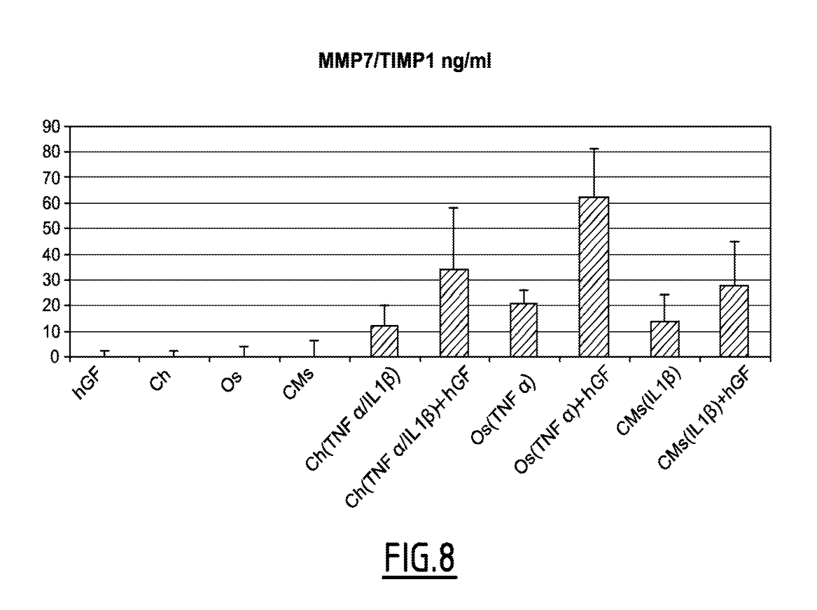

FIG. 9: Quantification of the MMP9/TIMP1 complex by ELISA (in ng/ml) in gingival fibroblasts (hGF), chondroblasts (Ch), osteoblasts (Os), striated muscle cells (CMs), chondroblasts stimulated by TNFα (10 ng/ml)+IL1β (5 ng/ml) alone (Ch(TNFα/IL1β) or co-cultured with hGF (Ch(TNFα/IL1β+hGF), osteoblasts stimulated by TNFα (10 ng/ml) alone (Os(TNFα)) or co-cultured with hGF (Os(TNFα)+hGF) and striated muscle cells stimulated by IL1β (5 ng/ml) alone (CMs(IL1β) or co-cultured with hGF (CMs(IL1β)+hGF).

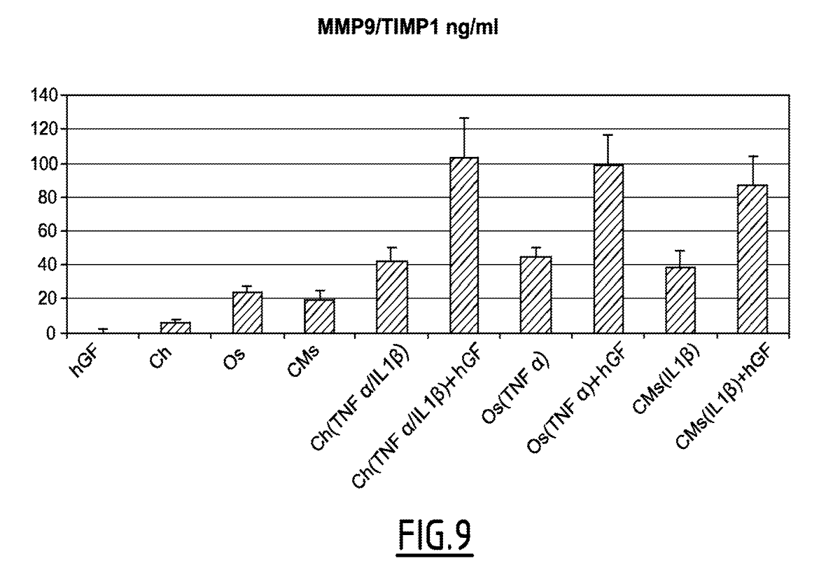

### EXAMPLE

This example aims at determining if human gingival fibroblasts inhibit the activities of 4 MMPs (MMP1, MMP3, MMP7, MMP9) overexpressed by three key cells involved in osteoarticular remodeling and orthopedic pathologies, in particular osteoarticular and muscular pathologies, namely: the cartilaginous cells (chondroblasts), the osteoblasts, and the muscle cells (striated muscle cells).

### Material and Methods

Key cells of osteoarticular remodeling have been used and cultured in vitro under inflammatory conditions:
Human chondrocytes (c-12710 Promocell)
Human osteoblasts (c-12760 Promocell)
Human striated muscle cells (c-12580 Promocell)

These cells were cultured in specific media (Promocell) for chondroblasts, osteoblasts and striated muscle cells. The cells were cultured in the lower part of transwells (Greiner bio-one, ref: 657 641). When confluence was reached, the cells were stimulated by a pro-inflammatory cytokine: TNFα (10 ng/ml) for osteoblasts, IL1β (5 ng/ml) for striated muscle cells, or an association TNFα (10 ng/ml)+IL1β (5 ng/ml) for chondroblasts during 24 h (Pretzel et al. (2009) Arthritis Res. Ther. 11:R25; Moran et al. (2009) Arthritis Res. Ther. 11:R113), to model an inflammatory environment and enable the expression of MMPs 1, 3, 7 and 9 as in pathological tissues.

After this stimulation, cells were then either co-cultured in DMEM medium (Dulbecco's Modified Eagle Medium) with gingival fibroblasts having reached confluence (upper part of the transwells), or cultured alone (control) during 24 h. The culture supernatants were analyzed after 24 h by ELISA to quantify the anti-inflammatory effect induced by human gingival fibroblasts (hGF) on chondroblasts (Ch), osteoblasts (Os) and striated muscle cells (CMs). The quantities of MMPs, TIMP1 (tissular inhibitor of all these MMPs) as well as MMP/TIMP1 complexes were quantified by ELISA (R&D).

### Results

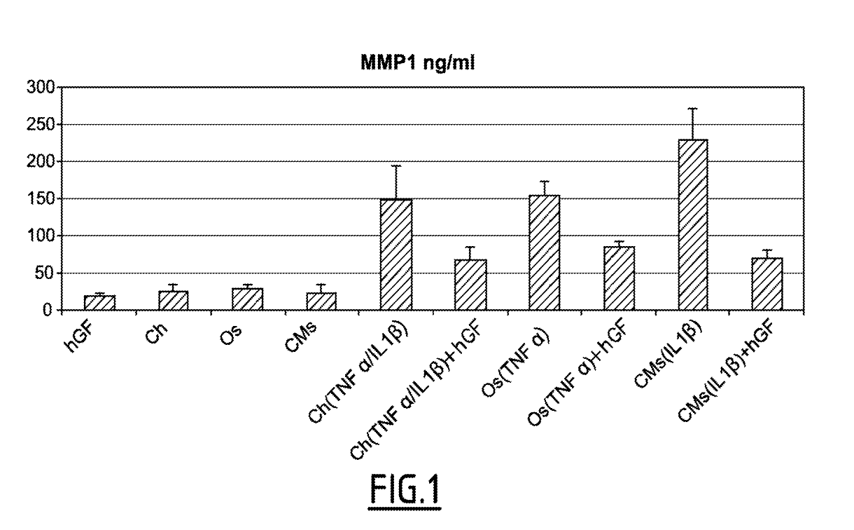

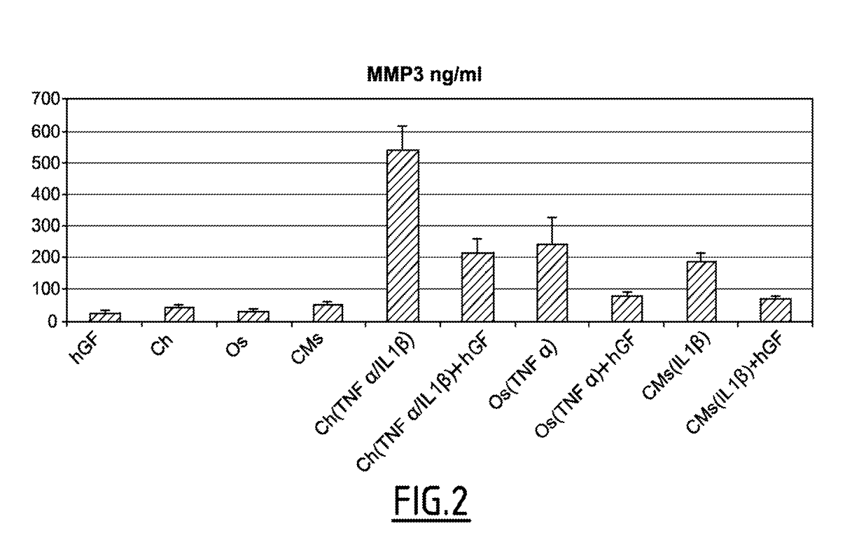

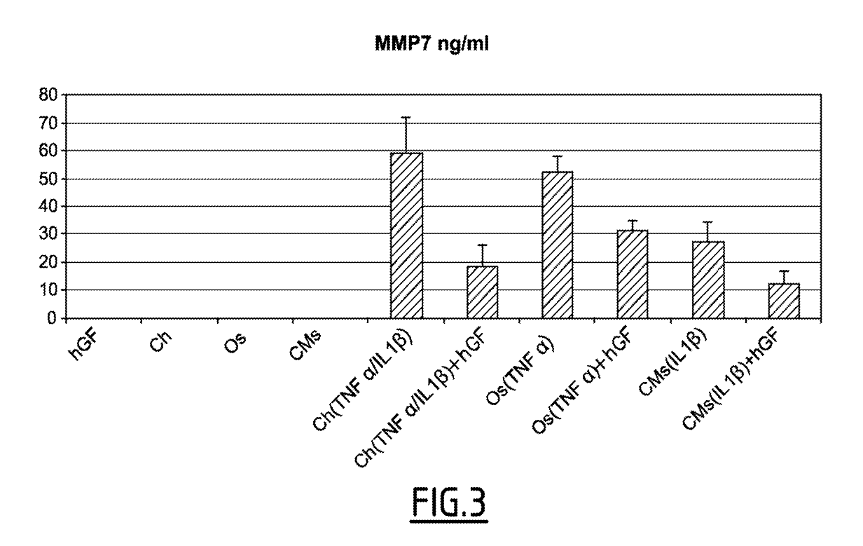

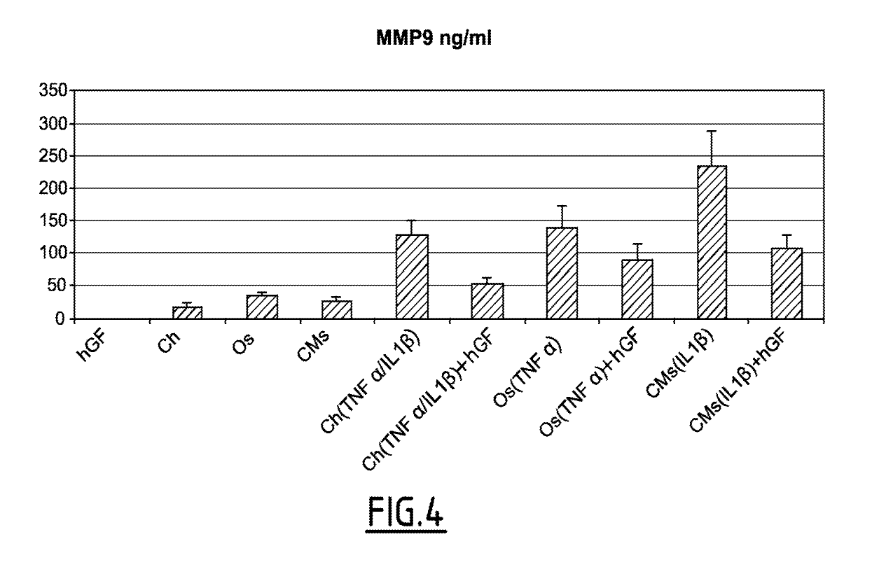

Stimulation of osteoblasts, striated muscle cells and chondroblasts by cytokines yielded an increased secretion of all MMPs (FIGS. 1 to 4). Stimulation of these three cellular types by cytokines thus models an inflammatory environment as in pathological tissues.

In co-culture with hGFs, it was observed that the concentrations of MMPs 1, 3, 7, 9 were lower to that of stimulated cells cultured alone, for all three cellular types (osteoblasts, chondroblasts and striated muscle cells) (FIGS. 1 to 4).

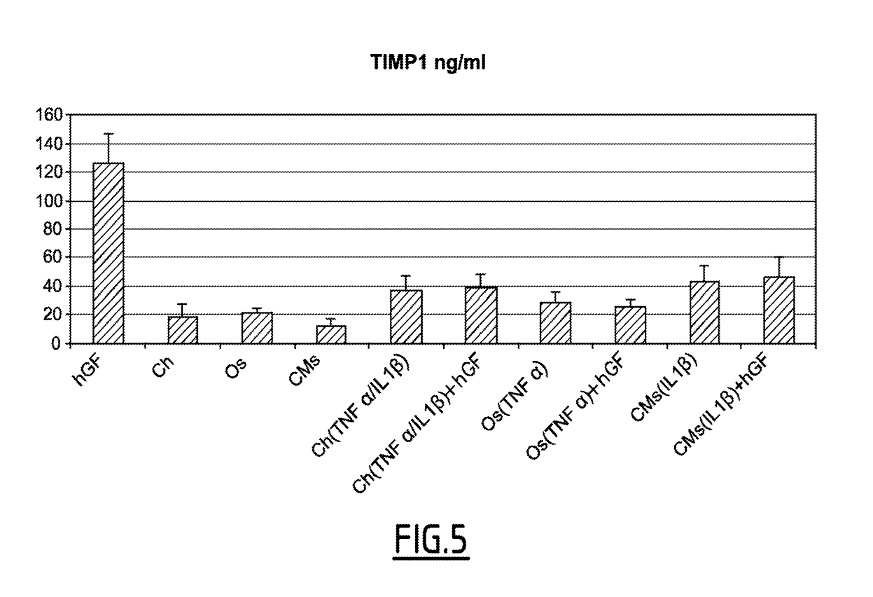

The quantification of TIMP1 in hGFs further showed that TIMP1 is strongly overexpressed in hGFs (FIG. 5).

It was also observed that the concentrations of the TIMP1/MMP1, TIMP1/MMP3, TIMP1/MMP7 and TIMP1/MMP9 complexes were increased in hGF co-cultures with respect to the culture supernatant of control cells. These results have thus shown that gingival fibroblasts, by overexpressing TIMP1, inhibit the activities of MMPs 1, 3, 7 and 9 secreted by chondroblasts, osteoblasts and striated muscle cells stimulated by pro-inflammatory cytokines.

These results thus demonstrate that gingival fibroblasts are capable of inhibiting the activity of MMPs in an environment similar to that of an orthopedic pathology, and that they are thus useful for treating such a pathology.

What is claimed is:

1. A method for the prevention or treatment of an orthopedic pathology of an individual, comprising administering a prophylactically or therapeutically effective amount of a gingival fibroblast-derived product to said individual wherein the fibroblast-derived product is selected from the group consisting of gingival fibroblast whole cells, a gingival fibroblast conditioned medium, a gingival fibroblast culture, and a gingival fibroblast extract.

2. The method according to claim 1, wherein the orthopedic pathology is selected from the group consisting of an osteoarticular orthopedic pathology, a muscular orthopedic pathology, a ligamentary orthopedic pathology and a tendinous orthopedic pathology.

3. The method according to claim 1, wherein the orthopedic pathology is an osteoarticular pathology.

4. The method according to claim 1, wherein the orthopedic pathology is an inflammatory osteoarticular pathology.

5. The method according to claim 1, wherein the orthopedic pathology is rheumatoid arthritis or osteoarthritis.

6. The method according to claim 1, wherein the gingival fibroblast-derived product is injected at a site of orthopedic defect of the individual.

7. The method according to claim 1, wherein the gingival fibroblast-derived product comprises gingival fibroblast whole cells.

8. The method according to claim 1, wherein the gingival fibroblast-derived product is a gingival fibroblast conditioned medium.

9. The method according to claim 1, wherein the gingival fibroblast-derived product is a gingival fibroblast culture.

10. The method according to claim 1, wherein the gingival fibroblast-derived product is a gingival fibroblast extract selected from the group consisting of a membrane extract, a cytoplasmic extract and a nuclear extract.

11. The method according to claim 1, wherein the gingival fibroblast-derived product is obtained from gingival fibroblasts which have not undergone a differentiation into cells having an osteogenic phenotype.

12. The method according to claim 1, wherein the gingival fibroblast-derived product is obtained from gingival fibroblasts taken from the individual.
